(19) United States
(12) Patent Application Publication
(10) Pub. No.: US 2009/0217393 A1
(43) Pub. Date: Aug. 27, 2009

(54) Growth Promoting Agent and Life Prolonging Agent

(75) Inventors: Masayuki Suzuki, Chita (JP); Kazunori Shinji, Handa (JP); Kazuo Ogasa, Tokai (JP); Tatsuya Uraji, Kawasaki (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: B FOOD SCIENCE CO., LTD., Aichi (JP)

(21) Appl. No.: 11/988,352

(22) PCT Filed: Jul. 26, 2006
(86) PCT No.: PCT/JP2006/314755

§ 371 Date: Feb. 5, 2009

(30) Foreign Application Priority Data
Jul. 27, 2005 (JP) 2005-216865

(51) Int. Cl. C07C 31/24; C12N 1/00; A01K 67/00; A01H 5/00; C12N 1/14

(52) U.S. Cl. 800/8; 568/852; 435/256.8; 800/298; 435/254.1; 435/243; 426/648

(57) ABSTRACT

An object of the present invention is to provide a growth promoting agent and a life prolonging agent which are safer than a conventional one, and can be used for stable and effective production of animals including farm animals, domestic fowls and fishes/shellfishes, stable and effective cultivation of plants including vegetables, fruits and grains, stable and effective culture of microorganisms, and stable and effective cultivation of mushrooms. The present invention provides the growth promoting agent comprising a sugar alcohol, particularly erythritol, as an active ingredient and a life prolonging agent comprising a sugar alcohol, particularly erythritol, as an active ingredient.

### TECHNICAL FIELD

The present invention relates to a growth promoting agent and a life prolonging agent which are used for breeding of animals, culture of microorganisms, and cultivation of plants and mushrooms. Moreover, the present invention relates to a feed, a nutritional supplement for animals, a fertilizer, a nutritional supplement for plants, a medium for microorganisms, a nutritional supplement for microorganisms, a medium for mushrooms, a nutritional supplement for mushrooms, a food, and a pharmaceutical composition, which contain the growth promoting agent and the life prolonging agent.

### BACKGROUND ART

There are required stable and effective production in production of farm animals, production of domestic fowls, production of fishes/shellfishes, cultivation of plants such as vegetables, fruits, and grains, and cultivation of mushrooms.

In order to accomplish the requirement, or in order to promote their growth or enhance their resistances against pests, there are employed a method involving enriching feeds, fertilizers and nutritional supplements with nutrients such as vitamins and amino acids, a method involving adding a digestive enzyme to the feeds, fertilizers, and nutritional supplements (see, for example, Patent Document 1), and a method involving adding antibiotics or growth hormones (see, for example, Patent Documents 2 and 3), etc.

However, by only the enhancement of vitamins or the like or the addition of a digestive enzyme, the above-mentioned conventional technologies cannot exhibit sufficient effects.

In addition, the use of antibiotics and growth hormones has a problem in safety.
Patent Document 1: JP 2593501 B
Patent Document 2: JP 2506568 B
Patent Document 3: JP 2952280 B

### DISCLOSURE OF THE INVENTION

### Problems to be Solved by the Invention

It is an object of the present invention to provide a growth promoting agent and a life prolonging agent, which can solve the problems of the conventional technologies, and are safer than the conventional ones, and can be used for stable and effective production of animals including farm animals, domestic fowls, and fishes/shellfishes, stable and effective cultivation of plants including vegetables, fruits, and grains, stable and effective culture of microorganisms, and stable and effective cultivation of mushrooms.

A further object of the present invention is to provide a feed, a nutritional. supplement for animals, a fertilizer, a nutritional supplement for plants, a medium for microorganisms, a nutritional supplement for microorganisms, a medium for mushrooms, a nutritional supplement for mushrooms, a food, and a pharmaceutical composition, which contain the growth promoting agent and the life prolonging agent.

### Means for Solving the Problems

The inventors of the present invention have made extensive studies to solve the above-mentioned problems and found out that, surprisingly, a growth promoting effect, a proliferative effect, a survival rate improving effect, a life prolonging effect, a repairing effect, etc. can be obtained by giving a sugar alcohol, in particular, erythritol to animals, plants, microorganisms, mushrooms, etc., and the present invention has been completed based on this findings.

The present invention according to Claim 1 provides a growth promoting agent or a life prolonging agent comprising a sugar alcohol as an active ingredient, which is used for breeding of animals, culture of microorganisms, and cultivation of plants and mushrooms.

The present invention according to Claim 2 provides the growth promoting agent or the life prolonging agent according to Claim 1, in which the sugar alcohol is erythritol.

The present invention according to Claim 3 provides a feed or a nutritional supplement for animals which is used for breeding animals, wherein the feed or the nutritional supplement for animals excellent in growth promoting effect or life prolonging effect is characterized by comprising erythritol in an amount of 0.1 to 6% by mass.

The present invention according to Claim 4 provides a fertilizer or a nutritional supplement for plants which is used for cultivating plants, wherein the fertilizer or the nutritional supplement for plants excellent in growth promoting effect or life prolonging effect for hydroponic cultivation is characterized by comprising erythritol in an amount of 0.01 to 3% by mass.

The present invention according to Claim 5 provides a fertilizer or a nutritional supplement for plants which is used for cultivating plants, wherein the fertilizer or the nutritional supplement for plants excellent in growth promoting effect or life prolonging effect for soil cultivation is characterized by comprising erythritol in an amount of 0.1 to 10% by mass.

The present invention according to Claim 6 provides a medium for microorganisms or nutritional supplement for microorganisms which is used for culturing microorganisms, wherein the medium for microorganisms or the nutritional supplement for microorganisms excellent in growth promoting effect or life prolonging effect is characterized by comprising erythritol in an amount of 0.001 to 10% by mass.

The present invention according to Claim 7 provides a medium for mushrooms or a nutritional supplement for mushrooms which is used for cultivating mushrooms, wherein the medium for mushrooms or the nutritional supplement for mushrooms excellent in growth promoting effect or life prolonging effect is characterized by comprising erythritol in an amount of 0.01 to 6% by mass.

The present invention according to Claim 8 provides a food excellent in growth promoting effect or life prolonging effect, characterized by comprising erythritol in an amount of 0.1 to 10% by mass.

The present invention according to Claim 9 provides a pharmaceutical composition excellent in growth promoting effect or life prolonging effect, characterized by comprising erythritol in an amount of 0.01 to 20% by mass.

The present invention according to Claim 10 provides an animal, a plant, a microorganism, or a mushroom which is obtained by giving one of the growth promoting agent, the life prolonging agent, the feed, the fertilizer, the medium, and the nutritional supplement according to any one of Claims 1 to 7 to promote the growth or to prolong the life.

### Effect of the Invention

According to the present invention, it is possible to provide a growth promoting agent that is safe and has an excellent growth promoting effect on: animals such as human, farm animals, domestic fowls, fishes/shellfishes, and pets; plants such as vegetables, fruits, ornamental plants; mushrooms; microorganisms, etc.

In addition, according to the present invention, it is possible to provide a life prolonging agent having a survival rate improving or life prolonging effect and a repairing effect on animals, plants, and mushrooms.

Moreover, according to the present invention, it is possible to provide a feed or a nutritional supplement for animals, a fertilizer or a nutritional supplement for plants, a medium for microorganisms or a nutritional supplement for microorganisms, a medium for mushrooms or a nutritional supplement for mushrooms, a food, and a pharmaceutical composition, which contain the growth promoting agent as described above and have the excellent growth promoting effect.

Further, according to the present invention, it is possible to provide a feed or a nutritional supplement for animals, a fertilizer or a nutritional supplement for plants, a medium for microorganisms or a nutritional supplement for microorganisms, a medium for mushrooms or a nutritional supplement for mushrooms, a food, and a pharmaceutical composition, which contain the life prolonging agent as described above and have the excellent life prolonging effect.

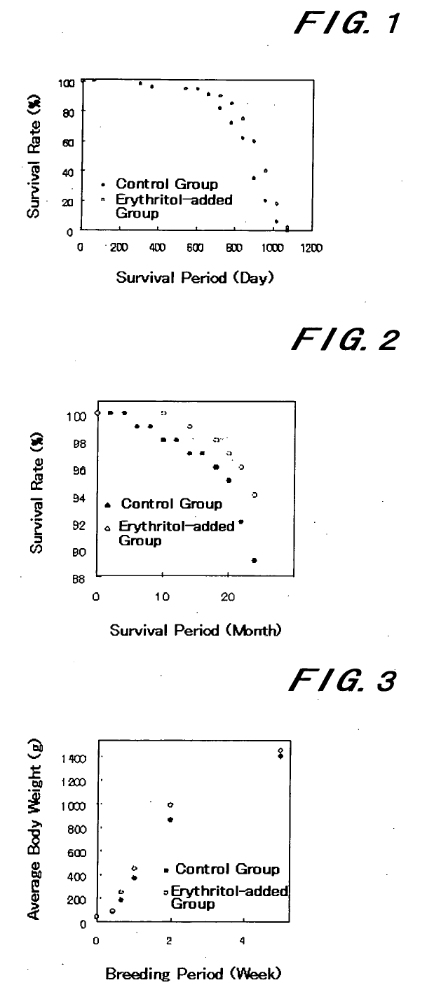

### BRIEF DESCRIPTION OF THE DRAWINGS

[FIG. 1] A diagram showing survival rates of mice in Example 1.

[FIG. 2] A diagram showing survival rates of layers in Example 2.

[FIG. 3] A diagram showing average body weights of layers in Example 2.

### BEST MODE FOR CARRYING OUT THE INVENTION

Hereinafter, the present invention will be described in detail.

The present invention according to Claim 1 is a growth promoting agent or a life prolonging agent comprising a sugar alcohol as an active ingredient.

The growth promoting agent according to Claim 1 of the present invention is an agent having an effect of promoting growth of animals including human, plants, microorganisms, and mushrooms and having an effect of enlarging the sizes, raising the growth rates, increasing the number, etc. compared with the case of normal growth of them.

The growth promoting effect is considered to be achieved by increasing number of cells, enlarging sizes of cells, inhibiting an effect of a growth inhibitory factor, etc.

For animals, the growth promoting agent has an effect of promoting weight gain or the like and an effect of promoting healthy growth of babyhood.

For plants, the growth promoting agent has preferable effects on the growth of plants such as: an effect of enlarging a partial or entire plant body by promoting growth of leaves, stems, or roots, by increasing sizes of fruits, bulbs, tubers, or flowers, or by increasing a number of flowers, a number of fruits, or a number of seeds; an effect of increasing germination rate of seeds; and an effect of promoting rooting of plants by cuttage, herbaceous cutting, or the like.

For microorganisms, the growth promoting agent has an effect of promoting proliferation of microorganisms or an effect of enlarging the sizes of microorganisms and enables stable production of fermented foods such as soy bean paste (miso), soy sauce, sake, and fermented soy beans (natto), amino acids, organic acids, nucleic acids, enzymes, vitamins, antibiotics, solvents including ethanol by using microorganisms.

For mushrooms, the growth promoting agent has an effect of promoting proliferation of mycelia, an effect of enlarging the sizes of basidiocarps, an effect of raising growth rate, and an effect of increasing the number.

The life prolonging agent according to Claim 1 of the present invention has an effect of prolonging average survival time and an effect of increasing the survival rate in the babyhood.

For animals such as farm animals and domestic fowls, the life prolonging agent has an effect of prolonging average survival times, an effect of increasing survival rates in the babyhood in breeding farm animals or the like, and an effect of repairing a wound site caused by an injury or a disease or a damage caused by a surrounding environment.

For fishes/shellfishes, the life prolonging agent has an effect of increasing egg hatch rate or survival rate in the babyhood.

For plants, the life prolonging agent has an effect of prolonging average survival time and an effect of prolonging a period when the plants have commercial values, for example, an effect of keeping commercial values of potted plants, cut flowers, vegetables, and fruits after harvests for longer periods of time, an effect of increasing the ratio of plants that grow healthily, and an effect of repairing a damage caused by a disease, wound, harmful insect, or surrounding environment.

For microorganisms, the life prolonging agent has an effect of increasing the number of survival microorganisms and enables stable production of fermented foods such as soy bean paste (miso), soy sauce, sake, and fermented soybeans (natto), amino acids, organic acids, nucleic acids, enzymes, vitamins, antibiotics, solvents including ethanol by using microorganisms.

For mushrooms, the life prolonging agent has an effect of keeping commercial values of mushrooms for longer periods of time, an effect of increasing the ratio of mushrooms that grow healthily, and an effect of repairing a damage caused by a disease, wound, harmful insect, or surrounding environment.

Examples of the animals include human, farm animals, domestic fowls, fishes/shellfishes, and pets.

Examples of the farm animals include the pig, cattle, horse, deer, goat, and sheep.

Examples of the pets include the dog, cat, rabbit, hamster, mouse, canary, and parakeet.

Examples of the domestic fowls include the chicken, game fowl, quail, duck, and turkey.

Examples of the fishes/shellfishes include: cultured marine products such as the trout, salmon, river eel, tuna, sea bream, yellowtail, flounder, globefish, shrimp, crab, shellfish, and other crustacea; and aquarium fishes such as goldfish, carp, and tropical fish.

Examples of the plants include: vegetables such as cabbage, cucumber, Chinese cabbage, komatsuna, radish, turnip, garlic, pumpkin, cauliflower, broccoli, burdock, carrot, spinach, garland chrysanthemum, balsam pear, celery, onion, tomato, eggplant, bell pepper, beansprouts, daikon sprouts, lettuce, balsam pear, green chive, asparagus, and okra; grains such as rice, wheat, barley, foxtail millet, barnyardmillet, common millet, buckwheat, and corn; tubers such as sweet potato, Irish potato, aroid, elephant foot, Chinese yam, yamato-imo, and wild yam; fruits such as orange, citrus iyo, navel orange, grapefruit, citrus sudachi, pineapple, loquat, grape, cherry, blue berry, prune, persimmon, Chinese quince, kiwifruit, ume apricot, strawberry, apple, apricot, fig, pear, melon, and watermelon; beans; herbs; ornamental plants such as rose, carnation, chrysanthemum, tulip, and zoysia; and pasture plants.

Examples of the microorganisms include: molds such as aspergilli and blue mold; basidiomycetes; yeasts; lactic acid bacteria; other bacteria; bifidobacteria; actinomycetes: and alga.

Examples of the mushrooms include shiitake, enokitake, bunashimeji, hon-shimeji, kuritake, tamogitake, mukitake, bunaharitake, yamabushitake, kikurage, shirokikurage, nameko, eryngii, common mushroom, truffle, mannentake, hatsutake, maitake, agaricus, oyster mushroom, and matsutake mushrooms.

The growth promoting agent or life prolonging agent according to Claim 1 of the present invention may be administered singly as a sugar alcohol serving as an active ingredient or administered simultaneously with another ingredient.

Examples of a method of administering the agents to animals include, but not limited to, oral administration, application, intravenous injection, intramuscular, subcutaneous, intradermal, intraperitoneal, intrarectal, ophthalmic, nasal, or sublingual administration. Examples of a method of administering the agents to plants include, but not limited to, absorption from root after soaked into soil, application, dispersion, and injection to leaves, stems, flowers, etc. Examples of a method of administering the agents to microorganisms include, but not limited to, addition to a medium to be used. Examples of a method of administering the agents to mushrooms include, but not limited to, addition to a medium to be used and spray to basidiocarps.

Examples of the forms of the growth promoting agent and life prolonging agent include liquid, powder, paste, pellet, capsule, granule, tablet, suspension, cataplasm, and adhesive plaster.

The sugar alcohol to be used in the growth promoting agent or life prolonging agent according to Claim 1 of the present invention may be one selected from sorbitol, mannitol, galactitol, xylitol, arabitol, ribitol, erythritol, threitol, inositol, maltitol, lactitol, cellobitol, palatinit, maltotriitol, and maltotetraitol, or a mixture of two or more of them.

Among the sugar alcohols, a preferable sugar alcohols is a monosaccharide sugar alcohol, and a more preferable sugar alcohol is erythritol as disclosed in Claim 2. Erythritol is commercially available as a calorie-free sweetener, but no report has been made on use of erythritol, which cannot be used as a source of calories, as a growth promoting agent and life prolonging agent.

In the present invention, the purity of erythritol is not particularly limited, but there may be used not only a purified product but also a fermentation solution containing unpurified erythritol, which is obtained in a production process of an ordinary fermentation method using a microorganism and using glucose as a material.

As disclosed in Claims 3 to 9, the present invention provides a feed, nutritional supplement for animals, fertilizer, nutritional supplement for plants, medium for microorganisms, nutritional supplement for microorganisms, medium for mushrooms, nutritional supplement for mushrooms, food, and pharmaceutical composition comprising erythritol. Moreover, as disclosed in Claim 10, the present invention provides an animal, plant, microorganism, or mushroom which is obtained by giving a growth promoting agent, life prolonging agent, feed, fertilizer, medium, or nutritional supplement.

The present invention according to Claim 3 provides a feed or nutritional supplement for animals having excellent growth promoting effect or life prolonging effect, which is characterized by comprising erythritol.

Such a feed or nutritional supplement for animals according to Claim 3 may be in the form of a general feed or nutritional supplement for animals, such as powder, granule, pellet, paste, or liquid, and is not particularly limited.

The feed or nutritional supplement for animals according to Claim 3 may contain an ingredient that can be generally added to a feed, such as corn flour, wheat bran, wheat, wheat flour, wheat germ, barley, defatted rice bran, rice, potato flour, soybean flour, soybean cake, rapeseed cake, sake cake, non-fat dry milk, fish flour, meat flour, meat-and-bone meal, fat and oil, vitamins (vitamin A, vitamin D, vitamin E, vitamin C, vitamin B1, vitamin B2, vitamin B6, vitamin B12, calcium pantothenate, folic acid, niacin, biotin, vitamin K, etc.), minerals (calcium carbonate, sodium phosphate, sodium chloride, iron sulfate, copper sulfate, zinc sulfate, magnesium sulfate, etc.). In addition, the feed or nutritional supplement for animals according to Claim 3 may optionally contain oligosaccharides, viable bacteria, polyphenols, plant extracts of herbs or the like, antioxidants, antibiotics, and growth hormones, etc.

The concentration of erythritol in the feed or nutritional supplement for animals according to Claim 3 varies depending on the species of animals to be fed with the feed or nutritional supplement for animals but is preferably 0.1 to 6% by mass. If the concentration is less than 0.1% by mass, its effect cannot be obtained, while if the concentration is more than 6% by mass, diarrhea may be induced by erythritol, both of which are not preferable. Note that, in the case of feeding of an extremely small amount of the feed or nutritional supplement for animals, the concentration of erythritol is preferably adjusted to more than 6% by mass, if necessary.

The present invention according to Claim 10 provides an animal which is obtained by giving the growth promoting agent, feed, or nutritional supplement for animals according to Claims 1 to 3 to promote the growth. The invention can increase the growth rate and enlarge the size, thereby providing an economically superior animal. For farm animals and domestic fowls, the invention is particularly preferable because of improvement of their production rates. In addition, the present invention according to Claim 10 provides an animal which is obtained by giving the life prolonging agent, feed, or nutritional supplement for animals according to Claims 1 to 3 to improve the survival rate and to have excellent life prolonging effect and repairing effect. For the resultant animal, the invention can increase the survival rate in the babyhood, thereby providing an economically superior animal, and in the case where the animal is a farm animal or domestic fowl, the invention can achieve healthy growth of the animal, thereby providing a texture or taste preferable as meat. Further, in the case where the animal is a farm animal or domestic fowl, the invention can reduce dosage level of a pharmaceutical, antibiotic, nutritional supplement for animals, feed, or the like, thereby providing a texture or taste preferable as meat.

The present invention according to Claim 4 or 5 provides a fertilizer or nutritional supplement for plants having excellent growth promoting effect or life prolonging effect, which is characterized by comprising erythritol.

Such a fertilizer or nutritional supplement for plants may be in the form of a general fertilizer such as powder, granule, pellet, paste, or liquid, and is not particularly limited.

The fertilizer or nutritional supplement for plants according to Claim 4 or 5 of the present invention includes fertilizers for hydroponic culture, nutritional supplements for hydroponic culture, soil improvement agents, seed treatment agents, seed coating agents, germination promoters, root stimulators, vital energy enhancing agents, activating agents, foliage treatment agents, etc.

The fertilizer or nutritional supplement for plants according to Claim 4 or 5 of the present invention may contain a component to be generally added to fertilizers or nutritional supplements for plants, such as nitrogen, phosphorus, potassium, or calcium.

The concentration of erythritol in the fertilizer or nutritional supplement for plants according to Claim 4 or 5 of the present invention varies depending on the species of a plant to be supplemented with the fertilizer or nutritional supplement for plants, but in the case of a liquid fertilizer for hydroponic culture or the like, the concentration of erythritol is preferably 0.01 to 3% by mass, while in the case of a fertilizer for soil, the concentration of erythritol is preferably 0.1 to 10% by mass, more preferably 0.5 to 5% by mass because the fertilizer is dispersed in soil. If the concentration is lower than the range, a desirable effect cannot be obtained, while if the concentration is higher than the range, growth of the plant is inhibited, and both of which are not preferable. Note that, in the case of a concentrated fertilizer or nutritional supplement for plants that is diluted before use or designed so as to be given to a plant in an extremely small amount and a delayed-type fertilizer or nutritional supplement for plants, it is necessary to increase the concentration of a sugar alcohol.

The present invention according to Claim 10 provides a plant which is obtained by giving the growth promoting agent, fertilizer, or nutritional supplement for plants according to Claim 1, 2, 4, or 5 to promote the growth. The invention can achieve short-time growth of a plant and enlarge the size of the plant compared with a plant grown with a normal fertilizer or nutritional supplement, thereby providing an economically superior plant. Moreover, the present invention according to Claim 10 provides a plant which is obtained by giving the life prolonging agent, fertilizer, or nutritional supplement for plants according to Claim 1, 2, 4, or 5 to improve the survival rate and to have excellent life prolonging effect and repairing effect. For the resultant plant, the invention can increase the survival rate in the babyhood, thereby providing an economically superior animal, and the invention can achieve healthy growth of the plant, thereby providing a texture or taste preferable as an edible plant. Moreover, the healthy growth of the plant can provide a plant rich in a functional component contained in plants. Further, the invention can reduce dosage levels of agrochemicals, nutritional supplements, and fertilizers, thereby providing a texture or taste preferable as an edible plant.

The present invention according to Claim 6 provides a medium for microorganisms or nutritional supplement for microorganisms having excellent growth promoting effect or life prolonging effect, which is characterized by comprising erythritol.

Such a medium for microorganisms or nutritional supplement for microorganisms comprising erythritol according to Claim 6 of the present invention may be in the form of a general medium for microorganisms or nutritional supplement for microorganisms, such as liquid or agar, and is not particularly limited.

The medium for microorganisms or nutritional supplement for microorganisms according to Claim 6 of the present invention may contain a component generally used for medium including: a carbon source such as starch, dextrin, or glucose; a nitrogen source such as urea, corn steep liquor, peptone, casein, yeast extract, or an ammonium salt; or a mineral such as a potassium salt, sodium salt, calcium salt, magnesium salt, phosphate, iron salt, zinc salt, copper salt, or manganese salt.

The concentration of erythritol in the medium for microorganisms or nutritional supplement for microorganisms according to Claim 6 of the present invention varies depending on the species of a microorganism to be cultured but is preferably 0.001 to 10% by mass. If the concentration is lower than the range, a desirable effect cannot be obtained, while if the concentration is higher than the range, growth of the microorganism is inhibited, both of which are not preferable. Note that, in the case of a nutritional supplement for microorganisms that is designed so as to be given to a microorganism in an extremely small amount, the concentration of a sugar alcohol is preferably adjusted to be higher than the above-mentioned range, if necessary.

The present invention according to Claim 10 provides a microorganism which is obtained by giving the growth promoting agent, medium for microorganisms, or nutritional supplement for microorganisms according to Claim 1, 2, or 6 to promote the growth. The invention can achieve short-time growth or proliferation compared with a microorganism grown in a normal medium, thereby providing an economically superior microorganism. In addition, the invention can enlarge the size, thereby providing a microorganism having excellent fermentation efficiency. Moreover, the present invention according to Claim 10 provides a microorganism which is obtained by giving the life prolonging agent, medium for microorganisms, or nutritional supplement for microorganisms according to Claim 1, 2, or 6 to improve the survival rate and to have excellent life prolonging effect and repairing effect. For the resultant microorganism, the invention can increase the survival rate, thereby providing an economically superior microorganism, and the invention can achieve healthy growth of the microorganism, thereby providing a microorganism having excellent fermentation efficiency.

The present invention according to Claim 7 provides a medium for mushrooms or nutritional supplement for mushrooms characterized by comprising erythritol.

The medium for mushrooms or nutritional supplement for mushrooms comprising erythritol according to Claim 7 may be in the form of a general medium or nutritional supplement, such as liquid, solid, agar, or wood and is not particularly limited.

In the present invention, the medium for mushrooms may contain a general medium component such as corn cob, sawdust, rice bran, wheat bran, bean curd refuse, corn bran, soybean cake, or rapeseed meal.

Meanwhile, the nutritional supplement for mushrooms may contain a general medium component including: a carbon source such as starch, dextrin, or glucose; a nitrogen source such as urea, corn steep liquor, peptone, casein, yeast extract, or an ammonium salt; or a mineral such as a potassium salt, sodium salt, calcium salt, magnesium salt, phosphate, iron salt, zinc salt, copper salt, or manganese salt.

The concentration of erythritol in the medium for mushrooms or nutritional supplement for mushrooms according to Claim 7 of the present invention varies depending on the species of mushrooms cultivated but is preferably 0.01 to 6% by mass. If the concentration is lower than the above-mentioned range, a desirable effect cannot be obtained, while the concentration is higher than the above-mentioned range, growth of a mushroom is inhibited, both of which are not preferable. Note that, in the case of a nutritional supplement for mushrooms designed so as to be given to a mushroom in an extremely small amount, the concentration of a sugar alcohol is preferably adjusted to be higher than the above-mentioned range, if necessary.

The present invention according to Claim 10 provides a mushroom which is obtained by giving the growth promoting agent, medium for mushrooms, or a nutritional supplement for mushrooms according to Claim 1, 2, or 7 to promote the growth. The invention can shorten the cultivation period of mushrooms, produce larger mushrooms, increase the number of the mushrooms by promoting germination of mushrooms, thereby providing an economically superior mushroom. Moreover, the present invention according to Claim 10 provides a mushroom which is obtained by giving the growth promoting agent, medium for microorganisms, or a nutritional supplement for microorganisms according to Claim 1, 2, or 7 to promote its growth, and can achieve healthy growth of the mushroom by its improvement of its survival rate and excellent life prolonging effect and repairing effect, thereby providing a mushroom having a texture or taste preferable as an edible mushroom. Meanwhile, the invention can achieve healthy growth of the mushroom, thereby providing a mushroom rich in a functional component contained in mushrooms. Moreover, the invention can reduce dosage level of the nutritional supplement or the like, thereby producing a mushroom having a texture or taste preferable as an edible mushroom.

The present invention according to Claim 8 provides a food having excellent growth promoting effect or life prolonging effect, which is characterized by comprising erythritol.

The food comprising erythritol according to Claim 8 of the present invention may be in the form of a general food including: meat products; fish products; milk products; seasonings; pickles; food boiled in soy source (tsukudani); fish cakes; table sweeteners; Japanese-style confections such as bean jam, sweet red bean jelly (yokan), and sweet bean paste bun (manju); breads; cakes; sweet stuffs such as creams, mousse, jellies, ice creams, yogurts, cookies, chocolates, candies, caramels, gums, and tablet confectioneries; and drinks such as soft drinks, carbonated drinks, juice drinks, coffee drinks, tea drinks, milk drinks, soybean milks, healthy drinks, and nutritional drink; and is not particularly limited.

In the present invention, the food may contain a component that can be generally added to foods, i.e., any of general food materials including: grains; fats and oils; tubers; beans, fruits; vegetables; mushrooms; eggs; milks; meats; fishes/shellfishes; seasonings; saccharides such as sugar, fructose, galactose, arabinose, xylose, glucose, mannose, lactose, palatinose, trehalose, glucosamine, and N-acetylglucosamine; oligosaccharides such as fructooligosaccharide, galactooligosaccharide, lactulose, raffinose, soybean oligosaccharide, isomaltooligosaccharide, maltooligosaccharide, xylooligosaccharide, cellooligosaccharide, agarooligosaccharide, chitin oligosaccharide, chitosan oligosaccharide, and cyclodextrin; dietary fibers such as polydextrose, resistant dextrin, cellulose, hemicellulose, glucomannan, apple fiber, corn fiber, beet fiber, pectin, chitin, and chitosan; aspartame, acesulfame K, suclarose, and saccharin, and salts thereof; sweeteners with high degree of sweetness, such as stevioside and a derivative thereof; citric acid, malic acid, acetic acid, lactic acid, and gluconic acid acidulants; vitamin such as vitamin C, B1, B2, B6, and B12, folic acid, pantothenic acid, biotin, niacin, nicotinamide, and vitamin A, D, and E; minerals such as inorganic salts and organic salts; natural pigments and artificial colorants; natural flavors and synthetic flavors; and functional components such as polyphenols, carnitine, coenzyme Q10, isoflavone, soybean proteins, soybean globulins, casein phosphopeptides, amino acids, peptides, herbs, and various plant extracts.

The concentration of erythritol in the food according to Claim 8 of the present invention varies depending on the type of the food, but it is preferably 0.1 to 10% by mass. If the concentration is lower than the above-mentioned range, a desirable effect cannot be obtained, while if the concentration is higher than the above-mentioned range, diarrhea may be induced to an animal (human) by the sugar alcohol, both of which are not preferable. Note that a food designed so as to be given in an extremely small amount is an exception.

The present invention according to Claim 9 provides a pharmaceutical composition having excellent growth promoting effect or life prolonging effect, which is characterized by comprising erythritol.

Such a pharmaceutical composition containing a growth promoting agent or life prolonging agent according to Claim 9 of the present invention may be in the form of a general drug or quasi drug, such as powder, granule, fine particle, powder, tablet, capsule, paste, liquid, syrup, drink, injection, drip, inhalant, spray, suppository, percutaneously absorbing agent, ophthalmic solution, adhesive plaster, or bath agent, and is not particularly limited.

In the present invention, the pharmaceutical composition may contain a general component that can be pharmaceutically added to a drug including drug components listed in the Japanese-Pharmacopoeia, additives listed in the drug-additive encyclopedia, vitamins (such as vitamin A, vitamin D, vitamin E, vitamin C, vitamin B1, vitamin B2, vitamin B6, vitamin B12, calcium pantothenate, folic acid, niacin, biotin, and vitamin K), and minerals. In addition, the pharmaceutical composition may optionally contain oligosaccharides, viable bacteria, polyphenols, plant extracts of herbs or the like, antioxidants, antibiotics, crude drug components, and other medicinal components.

The concentration of a sugar alcohol in the pharmaceutical composition according to Claim 9 of the present invention varies depending on the form or purpose, but it is preferably 0.01 to 20% by mass, more preferably 0.1 to 10% by mass. If the concentration is lower than the above-mentioned range, a desirable effect cannot be obtained, while if the concentration is higher than the above-mentioned range, diarrhea may be induced to an animal (human) by the sugar alcohol, both of which are not preferable. Note that a pharmaceutical composition designed so as to be given in an extremely small amount is an exception.

### EXAMPLES

Hereinafter, the present invention will be described in more detail byway of examples, but is not limited to the examples.

### Example 1

### Mouse Breeding Test

Crj:BDF1 mice (male) were divided into a group fed with a feed containing 5% by mass of erythritol (erythritol-added group; the present invention) and a group fed with a feed containing no erythritol (no-erythritol-added group; control), and bred, followed by investigation of 50% survival times, 75% survival times, average survival times, survival rates, and maximum survival times.

Table 1 shows the 50% survival times, 75% survival times, average survival times, and maximum survival times. In addition, the survival rates are shown in Table 1.

TABLE 1

| | | | Average | Maximum |
|---|---|---|---|---|
| | 50% survival | 75% survival | survival | survival |
| | time | time | time | time |
| No-erythritol- | 860 days | 776 days | 720 days | 1,020 days |
| added group | | | | |
| (control) | | | | |
| Erythritol- | 927 days | 850 days | 768 days | 1,080 days |
| added group | | | | |
| (the present | | | | |
| invention) | | | | |

The results revealed that the 50% survival time, 75% survival time, and average survival time of the erythritol-added group (the present invention) were prolonged compared with those of the no-erythritol-added group (control), and also revealed that the survival rate was prolonged. Note that there was no significant difference between the maximum survival times.

### Example 2

### Layer Growth Test

Layers (white Plymouth Rock) were divided into a group fed with a feed containing 5% by mass of erythritol (erythritol-added group; the present invention) and a group fed with a feed containing no erythritol (no-erythritol-added group; control), and bred. The breeding was performed for 24 months, which was generally defined so as to generate economic benefits, and the survival rates were continuously watched. The results are shown in FIG. 2.

Meanwhile, average body weights (g) were continuously watched for four weeks. The results are shown in FIG. 3.

The results revealed that both of the survival rate and average body weight of the erythritol-added group (the present invention) were increased compared with those of the no-erythritol-added group (control).

### Example 3

### Radish Sprout Cultivation Test

Radish sprouts were cultivated in 0.75% agar mediums containing erythritol, mannitol, and sorbitol each at concentrations of 0.05% by mass, 0.1% by mass, and 0.5% by mass as sugar alcohols, and in an agar medium containing no sugar alcohol (0%). 3 and 6 days after germination, the lengths of radish sprouts were measured. The results are shown in Table 2.

TABLE 2

| | Lengths (mm) of | |
|---|---|---|
| | radish sprouts | |
| Added sugar alcohol and amount thereof | After 3 days | After 6 days |
| No-sugar-alcohol-added group | 26 | 56 |
| Sugar alcohol-added group | | |
| 0.05% by mass erythritol added | 46 | 109 |
| 0.1% by mass erythritol added | 30 | 76 |
| 0.5% by mass erythritol added | 31 | 87 |
| 0.05% by mass mannitol added | 15 | 59 |
| 0.1% by mass mannitol added | 29 | 61 |
| 0.5% by mass mannitol added | 24 | 55 |
| 0.05% by mass sorbitol added | 30 | 68 |
| 0.1% by mass sorbitol added | 31 | 64 |
| 0.5% by mass sorbitol added | 27 | 52 |

The results revealed that the radish sprouts of the sugar alcohol-added group were significantly longer than those of the no-sugar-alcohol-added group. Of those, the erythritol-added groups were found to have particularly excellent growth promoting effects within a wide range of concentrations.

### Example 4

### Arabidopsis Plant Cultivation Test

Arabidopsis plants were separately inoculated into a Murashige & Skoog medium containing 0.06% by mass of erythritol (erythritol-added group; the present invention) and a medium containing no erythritol (no-erythritol-added group; control), followed by comparison of growth of the Arabidopsis plants.

As a result, in the case of the erythritol-added group (the present invention), growth promoting effect on both of the roots and the leaves/stems of the Arabidopsis plants was confirmed compared with the no-erythritol-added group (control).

### Example 5

### Yeast Culture Test

A yeast Saccharomyces cerevisiae was cultured in an SD medium containing 0.006% by mass of erythritol (erythritol-added group; the present invention) and in an SD medium containing no erythritol (no-erythritol-added group; control). 27 hours later, absorbances at 660 nm were measured.

As a result, the absorbance of the no-erythritol-added group (control) was found to be 2.65, while the absorbance of the erythritol-added group (the present invention) was found to be 2.88.

Therefore, yeast proliferation effect was confirmed in the erythritol-added group (the present invention).

Meanwhile, comparison of yeast cells under a microscope revealed that the sizes of the yeast cells of the erythritol-added group (the present invention) were larger by about 20% than those of the no-erythritol-added group (control).

### Example 6

### Mushroom Cultivation Test

A shiitake cultivation test was performed using a commercially available shiitake cultivation kit.

The cultivation kit is a product produced by hardening wood chips prepared by pulverizing broad-leaved tree, together with a nutritional supplement and a shiitake fungus into a block form. The blocks were separately immersed in aqueous solutions containing 0% by mass (no addition), 0.05% by mass, 0.5% by mass, and 5% by mass of erythritol, and the surfaces of the blocks were washed. Then, the blocks were placed in plastic bags to prevent drying, and the tops of the bags were closed with clothespins, followed by cultivation of the shiitake while adjusting the temperature to 23° C. during the daytime and to 8° C. during the nighttime. Whenever the surfaces of the blocks were dried, the surfaces of the blocks were moisturized using a spray with the aqueous solutions containing erythritol at the above-mentioned each concentration. 3 days later, the shiitake started to germinate on the blocks, and 8 days later, the diameter and number of each pileus of the shiitake were measured. The results are shown in Table 3.

TABLE 3

| | Number of mushroom |
|---|---|
| Added erythritol and amount | with pileus diameter |
| thereof | of 5 cm or more |
| No-erythritol-added group | 2 |
| Erythritol-added group | |
| 0.05% by mass erythritol added | 8 |
| 0.5% by mass erythritol added | 5 |
| 5.0% by mass erythritol added | 9 |

As a result, the numbers of mushrooms with a pileus diameter of 5 cm or more, which is a size preferable for eating, of the erythritol-added groups (the present invention) were found to significantly increase at all the concentrations compared with the no-erythritol-added group (control).

### INDUSTRIAL APPLICABILITY

The growth promoting agent and life prolonging agent of the present invention contain, as an active ingredient, a sugar alcohol that is regularly taken as a sweetener, and therefore, they are safe and can be used for breeding animals, cultivating plants, culturing microorganisms, and cultivating mushrooms.

1. A growth promoting agent or a life prolonging agent, comprising a sugar alcohol as an active ingredient, which is used for breeding of animals, culture of microorganisms, and cultivation of plants and mushrooms.

2. The growth promoting agent or a life prolonging agent according to claim 1, wherein the sugar alcohol is erythritol.

3. A feed or a nutritional supplement for animals which is used for breeding animals, wherein the feed or the nutritional supplement for animals excellent in growth promoting effect or life prolonging effect is characterized by comprising erythritol in an amount of 0.1 to 6% by mass.

4. A fertilizer or a nutritional supplement for plants which is used for cultivating plants, wherein the fertilizer or the nutritional supplement for plants excellent in growth promoting effect or life prolonging effect for hydroponic cultivation is characterized by comprising erythritol in an amount of 0.01 to 3% by mass.

5. A fertilizer or a nutritional supplement for plants which is used for cultivating plants, wherein the fertilizer or the nutritional supplement for plants excellent in growth promoting effect or life prolonging effect for soil cultivation is characterized by comprising erythritol in an amount of 0.1 to 10% by mass.

6. A medium for microorganisms or a nutritional supplement for microorganisms which is used for culturing microorganisms, wherein the medium for microorganisms or the nutritional supplement for microorganisms excellent in growth promoting effect of life prolonging effect is characterized by comprising erythritol in an amount of 0.001 to 10% by mass.

7. A medium for mushrooms or a nutritional supplement for mushrooms which is used for cultivating mushrooms, where in the medium for mushrooms or the nutritional supplement for mushrooms excellent in growth promoting effect or life prolonging effect is characterized by comprising erythritol in an amount of 0.01 to 6% by mass.

8. A food excellent in growth promoting effect or life prolonging effect, characterized by comprising erythritol in an amount of 0.1 to 10% by mass.

9. A pharmaceutical composition excellent in growth promoting effect or life prolonging effect, characterized by comprising erythritol in an amount of 0.01 to 20% by mass.

10. An animal, plant, microorganism, or mushroom which is obtained by giving one of the growth promoting agent, the life prolonging agent, the feed, the fertilizer, the medium, and the nutritional supplement according to any one of claims 1 to 7 to promote the growth or to prolong the life.
